(19) United States
(12) Patent Application Publication
(10) Pub. No.: US 2008/0119414 A1
(43) Pub. Date: May 22, 2008

(54) Mixtures Of Quaternary And Non-Ionic Surfactants

(76) Inventors: Ansgar Behler, Bottrop (DE); Frank Clasen, Hilden (DE)

Correspondence Address: COGNIS CORPORATION;PATENT DEPARTMENT, 300 BROOKSIDE AVENUE, AMBLER, PA, 19002, US

(21) Appl. No.: 11/718,527

(22) PCT Filed: Oct. 27, 2005
(86) PCT No.: PCT/EP05/11482

§ 371 Date: May 3, 2007

(30) Foreign Application Priority Data
Nov. 5, 2004 (DE) 10 2004 054 035.7

(51) Int. Cl. A61K 8/60; C07H 15/04; C11D 1/62

(52) U.S. Cl. 514/25; 536/17.9; 510/470

(57) ABSTRACT

The invention relates to a composition containing an alkyl and/or alkenyl oligoglycoside and a cationic alkyl and/or alkenyl oligoglycoside of the formula:

R²—O-[G]ₓ-Z_{y} (II)

wherein, R² is a linear or branched alkyl and/or alkenyl group containing 4 to 22 carbon atoms, G is a sugar unit containing 5 or 6 carbon atoms, x is a number of 1 to 10, y is a number of 1 to 4 and Z is an —OCH₂—(CH(B))ᵥ—(CH(A))_{w}-N⁺(R³R⁴R⁵)X⁻ group, where A and B independently of one another represent hydrogen or a hydroxyl group, v and w independently of one another are numbers of 1 to 18, X is an anion selected from the group consisting of chloride, bromide, iodine, fluoride, NO₃⁻, SO₄²⁻ and PO₄³⁻, and R³, R⁴ and R⁵ independently of one another represent hydrogen or alkyl and/or alkenyl groups containing 1 to 22 carbon atoms and at least two of the substituents R³ to R⁵ are alkyl and/or alkenyl groups. The composition is distinguished by high mildness towards the skin and hair and, at the same time, by excellent foam properties and cleaning performance.

### RELATED APPLICATIONS

This application is filed under 35 U.S.C. § 371 claiming priority from application PCT/EP2005/011482 filed Nov. 27, 2005, which claims priority from German application DE 10 2004 054 035.7 filed Nov. 5, 2004; the entire contents of each application are incorporated herein by reference.

### FIELD OF THE INVENTION

This invention relates generally to cationically substituted glycoside surfactants and, more particularly, to a process for their production and to their use in the field of cosmetics and cleaning preparations.

### BACKGROUND OF THE INVENTION

By virtue of their extreme mildness, surfactants based on natural raw materials, more particularly on saccharides, enjoy great popularity. Accordingly, there is considerable interest in finding derivatives of this class of compounds which, aside from new properties, continue to be mild. Thus, cationic derivatives of alkyl polyglycosides are the subject of WO 90/15809. The quaternary surfactants according to WO 90/15809 are produced by alkylation of quaternized saccharides because the quaternization of alkyl polyglycosides does not work.

The disadvantage of this process lies in the starting products for the production of the cationic derivatives of the alkyl polyglycosides. Alkyl polyglycosides are a common and frequently used class of surfactants which could therefore easily be used as starting material for the derivatization. Unreacted educt could then remain in the product and contribute to its surface-active properties.

Accordingly, the problem addressed by the present invention lay in the simple production of cationic alkyl polyglycoside derivatives where there would be no formation of harmful secondary products that would have to be removed. In addition, the invention set out to provide a surfactant mixture based on highly dermatologically compatible sugar surfactants which would have improved foam and emulsification properties.

### BRIEF DESCRIPTION OF THE INVENTION

The present invention relates to preparations containing;
(a) alkyl and/or alkenyl oligoglycosides corresponding to formula (I):

R¹O-[G]ₚ (I)
in which R¹ is a linear or branched alkyl and/or alkenyl group containing 4 to 22 carbon atoms, G is a sugar unit containing 5 or 6 carbon atoms and p is a number of 1 to 10, and
(b) cationic alkyl and/or alkenyl oligoglycoside derivatives corresponding to formula (II):

R²—O-[G]ₓ-Z_{y} (II)
in which R² is a linear or branched alkyl and/or alkenyl group containing 4 to 22 carbon atoms, G is a sugar unit containing 5 or 6 carbon atoms, x is a number of 1 to 10, y is a number of 1 to 4 and Z is an —OCH₂—(CH(B))ᵥ—(CH(A))_{w}-N⁺(R³R⁴R⁵)X⁻ group, where A and B independently of one another represent hydrogen or a hydroxyl group, v and w independently of one another are numbers of 1 to 18, X is an anion selected from the group consisting of chloride, bromide, iodine, fluoride, NO₃⁻, SO₄²⁻ and PO₄³⁻, and R³, R⁴ and R⁵ independently of one another represent hydrogen or alkyl and/or alkenyl groups containing 1 to 22 carbon atoms and at least two of the substituents R³ to R⁵ are alkyl and/or alkenyl groups.

These preparations are distinguished by high mildness towards the skin and hair and, at the same time, by excellent foam properties and cleaning performance.

### DETAILED DESCRIPTION OF THE INVENTION

Components (a) and (b) are preferably present in a ratio by weight of 10:90 to 90:10 and, in a particularly preferred embodiment, are present in a ratio by weight of 40:60 to 60:40.

In a preferred embodiment, component (b) is selected from compounds of formula (II) where B is hydrogen, A is a hydroxy group and w and v stand for 1, R³ and R⁴ stand for methyl, R⁵ stands for a methyl or lauryl group and X⁻ stands for chloride. In a particularly preferred embodiment, component (b) is selected from compounds of formula (II) where B is hydrogen, A is a hydroxy group, w and v stand for 1, R³, R⁴ and R⁵ represent a methyl group and X⁻ stands for chloride.

The preparations according to the invention are produced by a so-called one-pot process. Unreacted alkyl and/or alkenyl oligoglycosides remain in the end product. Accordingly, the present invention relates to a process for the production of cationic alkyl and/or alkenyl oligoglycoside derivatives corresponding to formula (II):

R²—O-[G]ₓ-Z_{y} (II)

in which R² is a linear or branched alkyl and/or alkenyl group containing 4 to 22 carbon atoms, G is a sugar unit containing 5 or 6 carbon atoms, x is a number of 1 to 10, y is a number of 1 to 4 and Z is an —OCH₂—(CH(B))ᵥ—(CH(A))_{w}-N⁺(R³R⁴R⁵)X⁻ group, where A and B independently of one another represent hydrogen or a hydroxyl group, v and w independently of one another are numbers of 1 to 18, X is an anion selected from the group consisting of chloride, bromide, iodine, fluoride, NO₃⁻, SO₄²⁻ and PO₄³⁻, and R³, R⁴ and R⁵ independently of one another represent hydrogen or alkyl and/or alkenyl groups containing 1 to 22 carbon atoms and at least two of the substituents R³ to R⁵ are alkyl and/or alkenyl groups, characterized in that alkyl and/or alkenyl oligoglycosides are reacted with quaternizing agents corresponding to formula (III):

Cl—CH₂—(CH(B))ᵥ—(CH(A))_{w}-N⁺(R³R⁴R⁵)X⁻ (III)

in which A, B, v, w, R³, R⁴ and R⁵ have the same meaning as in formula (II), in the presence of strong bases.

The starting products preferably used are alkyl and/or alkenyl oligoglycosides corresponding to formula (I):

R¹O-[G]ₚ (I)

in which R¹ is a linear or branched alkyl and/or alkenyl group containing 4 to 22 carbon atoms, G is a sugar unit containing 5 or 6 carbon atoms and p is a number of 1 to 10. The quaternizing agent of formula (III) is preferably selected from compounds where B is hydrogen, A is a hydroxy group, w and v stand for 1, R³, R⁴ and R⁵ represent a methyl group and X⁻ is chloride.

Alkyl and alkenyl oligoglycosides are known nonionic surfactants corresponding to formula (I):

R¹O-[G]ₚ (I)

in which R¹ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (I) indicates the degree of oligomerization (DP, i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.5 are preferred from the applicational point of view. The alkyl or alkenyl radical R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R¹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated Cl_{12/14} cocoalcohol with a DP of 1 to 3 are preferred.

A preferred embodiment of the process according to the invention is characterized in that the alkyl and/or alkenyl oligoglycosides and the quaternizing agents of formula (III) are used in a molar ratio of 1:0.5 to 1:4 and, more particularly, in a molar ratio of 1:1 to 1:1.5.

Particularly good yields are obtained when the reaction is carried out at temperatures of 20 to 100° C. and, as in a particularly preferred embodiment, at temperatures of 40 to 80° C.

The surfactant preparations obtained by this process, which contain a mixture of alkyl and/or alkenyl oligoglycosides of formula (I) in combination with cationic alkyl and/or alkenyl oligoglycoside derivatives of formula (II), may be used in combination with other standard ingredients, such as, for example, other surfactants, emulsifiers, oil components, active principles, silicones, fats and waxes, etc., in cosmetic preparations, such as shampoos and shower baths, or in cleaning preparations. However, the preparations according to the invention may also be applied to cleaning cloths either directly or after processing to cosmetic preparations by addition of other standard ingredients. Such cleaning cloths are known as dry or wet wipes according to the water content of the impregnated cloths. These wipes may be used either directly or after moistening for the cleansing or conditioning of the skin and hair.

### EXAMPLES

### 1. APG-QUAT

### QUAB 188

In a 1000 ml three-necked flask, 21.2 g NaOH microprills (0.53 mol) and 238 g isopropanol were added to 328.1 g (0.4 mol) Plantacare 1200 and the whole was reacted with 108.4 g (0.4 mol) QUAB 188 with stirring at 60° C. The reaction was terminated after 1 h when the theoretical quantity of chloride (4.16% Cl⁻) had been reached. The product was a yellow homogeneous liquid.

| | |
|---|---|
| Dry residue: | 38.7% by weight |
| of which Plantacare 1200: | 13.5% by weight |
| QUAB 188 | <0.1% by weight |

### 2. APG-QUAT

### QUAB 342

In a 500 ml three-necked flask, 11.7 g NaOH microprills (0.29 mol) were added to 164.4 g (0.2 mol) Plantacare 1200 and the whole was reacted with 222.3 g (0.26 mol) QUAB 342 with stirring at 80° C. The reaction was terminated after 1 h when the theoretical quantity of chloride (4.85% Cl⁻) had been reached. The product was yellow, inhomogeneous and viscous.

| | |
|---|---|
| Dry residue: | 49.1% by weight |
| of which Plantacare 1200: | 6.8% by weight |
| QUAB 342 | <0.1% by weight |

### 3. APG-QUAT

### QUAB 360

In a 500 ml three-necked flask, 10.6 g NaOH microprills (0.265 mol) were added to 164.4 g (0.2 mol) Plantacare 1200 and the whole was reacted with 180.5 g (0.2 mol) QUAB 360 with stirring at 80° C. The reaction was terminated after 1 h when the theoretical quantity of chloride (4.05% Cl⁻) had been reached. A yellow, inhomogeneous and viscous product was obtained after dilution with 113.1 g water.

| | |
|---|---|
| Dry residue: | 35.5% by weight |
| of which Plantacare 1200: | 6.8% by weight |

### 4. APG-QUAT

### QUAB 426

In a 500 ml three-necked flask, 10.6 g NaOH microprills (0.265 mol) were added to 164.4 g (0.2 mol) Plantacare 1200 and the whole was reacted with 217.3 g (0.2 mol) QUAB 426 with stirring at 80° C. The reaction was terminated after 1 h when the theoretical quantity of chloride (4.05% Cl⁻) had been reached. A yellow, inhomogeneous and viscous product was obtained after dilution with 114.9 g water.

| | |
|---|---|
| Dry residue: | 46.2% by weight |
| of which Plantacare 1200: | 6.7% by weight |

We claim:

1-11. (canceled)

12. A composition comprising:
(a) an alkyl and/or alkenyl oligoglycoside of the formula:
R¹O-[G]ₚ (I),
wherein, R¹ is a linear or branched alkyl and/or alkenyl group containing 4 to 22 carbon atoms, G is a sugar residue containing 5 or 6 carbon atoms and p is a number of 1 to 10, and
(b) a cationic alkyl and/or alkenyl oligoglycoside derivative of the formula:
R²—O-[G]ₓ-Z_{y} (II),
wherein, R² is a linear or branched alkyl and/or alkenyl group containing 4 to 22 carbon atoms, G is a sugar unit containing 5 or 6 carbon atoms, x is a number of 1 to 10, y is a number of 1 to 4 and Z is an —OCH₂—(CH(B))ᵥ—(CH(A))_{w}-N⁺(R³R⁴R⁵)X⁻ group, where A and B independently of one another represent hydrogen or a hydroxyl group, v and w independently of one another are numbers of 1 to 18, X is an anion selected from the group consisting of chloride, bromide, iodine, fluoride, NO₃⁻, SO₄²⁻ and PO₄³⁻, and R³, R⁴ and R⁵ independently of one another represent hydrogen or alkyl and/or alkenyl groups containing 1 to 22 carbon atoms and at least two of the substituents R³ to R⁵ are alkyl and/or alkenyl groups.

13. The composition of claim 12, wherein, (a) and (b) are present in a ratio by weight of 10:90 to 90:10.

14. The composition of claim 12, wherein, in component (b) of formula (II), B is hydrogen, A is a hydroxyl group, w and v are 1, R³ and R⁴ are methyl, R⁵ is a methyl or a lauryl group and X is chloride.

15. The composition of claim 12, wherein, in component (b) of formula (II), B is hydrogen, A is a hydroxyl group, w and v are 1, R³, R⁴ and R⁵ are methyl groups and X⁻ is chloride.

16. A process for the production of cationic alkyl and/or alkenyl oligoglycoside derivatives of the formula:
R²—O-[G]ₓ-Z_{y} (II),
wherein, R² is a linear or branched alkyl and/or alkenyl group containing 4 to 22 carbon atoms, G is a sugar residue containing 5 or 6 carbon atoms, x is a number of 1 to 10, y is a number of 1 to 4 and Z is an —OCH₂—(CH(B))ᵥ—(CH(A))_{w}-N⁺(R³R⁴R⁵)X⁻ group, where A and B independently of one another represent hydrogen or a hydroxyl group, v and w independently of one another are numbers of 1 to 18, X is an anion selected from the group consisting of chloride, bromide, iodine, fluoride, NO₃⁻, SO₄²⁻ and PO₄³⁻, and R³, R⁴ and R⁵ independently of one another represent hydrogen, alkyl and/or alkenyl groups containing 1 to 22 carbon atoms and at least two of R³ to R⁵ are alkyl and/or alkenyl groups, wherein, an alkyl and/or alkenyl oligoglycoside is reacted with a quaternizing agent of the formula:
Cl—CH₂—(CH(B))ᵥ—(CH(A))_{w}—N⁺(R³R⁴R⁵)X⁻ (III)
in which A, B, v, w, R³, R⁴ and R⁵ have the same meaning as in formula (II), in the presence of a strong base.

17. The process of claim 16, wherein, the alkyl and/or alkenyl oligoglycoside and the quaternizing agent of formula (III) are used in a molar ratio of 1:0.5 to 1:4.

18. The process of claim 16, wherein, the alkyl and/or alkenyl oligoglycoside and the quaternizing agent of formula (III) are used in a molar ratio of 1:1 to 1:1.5.

19. The process of claim 16, wherein, the oligoglycoside and quaternizing agent are reacted at a temperature in a range of 40 to 80° C.

20. The process of claims 16, wherein, in formula (III), B is hydrogen, A is a hydroxyl group, w and v are 1, R³, R⁴ and R⁵ are a methyl group and X⁻ is chloride.

21. A cosmetic or cleaning preparation comprising the composition of claim 16.

22. A wipe comprising the composition of claim 16.

23. The composition of claim 13, wherein, in component (b) of formula (II), B is hydrogen, A is a hydroxyl group, w and v are 1, R³ and R⁴ are methyl, R⁵ is a methyl or a lauryl group and X is chloride.

24. The composition of claim 13, wherein, in component (b) of formula (II), B is hydrogen, A is a hydroxyl group, w and v are 1, R³, R⁴ and R⁵ are methyl groups and X⁻ is chloride.

25. The composition of claim 14, wherein, in component (b) of formula (II), B is hydrogen, A is a hydroxyl group, w and v are 1, R³, R⁴ and R⁵ are methyl groups and X⁻ is chloride.

26. The process of claim 17, wherein, the alkyl and/or alkenyl oligoglycoside and the quaternizing agent of formula (III) are used in a molar ratio of 1:1 to 1:1.5.

27. The process of claim 17, wherein, the oligoglycoside and quaternizing agent are reached at a temperature in a range of 40 to 80° C.

28. The process of claim 18, wherein, the oligoglycoside and quaternizing agent are reacted at a temperature in a range of 40 to 80° C.

29. The process of claim 17, wherein, in formula (III), B is hydrogen, A is a hydroxyl group, w and v are 1, R³, R⁴ and R⁵ are a methyl group and X⁻ is chloride.

30. The process of claim 18, wherein, in formula (III), B is hydrogen, A is a hydroxyl group, w and v are 1, R³, R⁴ and R⁵ are a methyl group and X⁻ is chloride.

31. The process of claim 19, wherein, in formula (III), B is hydrogen, A is a hydroxyl group, w and v are 1, R³, R⁴ and R⁵ are a methyl group and X⁻ is chloride.
